(19) United States
(12) Patent Application Publication
(10) Pub. No.: US 2025/0268509 A1
(43) Pub. Date: Aug. 28, 2025

(54) NEUROFEEDBACK SYSTEM, BRAIN-STATE DETERMINATION AND REPORTING SYSTEM AND METHODS FOR USE THEREWITH

(71) Applicant: Metiris ApS, Taastrup (DK)

(72) Inventors: Soren Bo Andersen, Taastrup (DK); Lars Henriksen, Bronshoj (DK); Georgios N. Yannakakis, Sliema (MT); Lukasz Cygan, Maslomiaca (PL); Philip Michael Zeman, Shawnigan Lake (CA)

(73) Assignee: Metiris ApS, Taastrup (DK)

(21) Appl. No.: 19/021,537

(22) Filed: Jan. 15, 2025

Related U.S. Application Data
(60) Provisional application No. 63/556,973, filed on Feb. 23, 2024.

(51) Int. Cl. A61B 5/375; A61B 5/00; A61B 5/384

(52) U.S. Cl. CPC A61B 5/375; A61B 5/384; A61B 5/7267

(57) ABSTRACT

A system operates by: sending gamified neurofeedback displays for display via a graphical user interface of a client device and receiving client device interactions with the graphical user interface from the client device; receiving neurosensing device signals via at least one neurosensing device corresponding to a user of the client device; preprocessing and filtering the neurosensing device signals to reduce artifacts and to produce filtered signals corresponding to a plurality of different brain waves of the user; generating frequency and time analysis data based on the filtered signals; extracting feature data based on the frequency and time analysis data; generating, via an artificial intelligence (AI) neuro-classification engine trained via machine learning, neuro-classification data based on the feature data, generating brain assessment data based on the neuro-classification data, generating, via at least one gaming application, the gamified neurofeedback displays based on client device interactions and/or generating neurofeedback results based on the neuro-classification data.

### CROSS-REFERENCE TO RELATED APPLICATIONS

The present U.S. Utility Patent Application claims priority pursuant to 35 U.S.C. § 119(e) to U.S. Provisional Application No. 63/556,973, entitled “NEUROFEEDBACK SYSTEM, BRAIN-STATE DETERMINATION AND REPORTING SYSTEM AND METHODS FOR USE THEREWITH”, filed Feb. 23, 2024, which is hereby incorporated herein by reference in its entirety and made part of the present U.S. Utility Patent Application for all purposes.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

### Technical Field of the Invention

The disclosed subject matter relates to computer systems and devices for analyzing brain states and providing neurofeedback.

BRIEF DESCRIPTION OF THE SEVERAL VIEWS OF THE DRAWING(S)

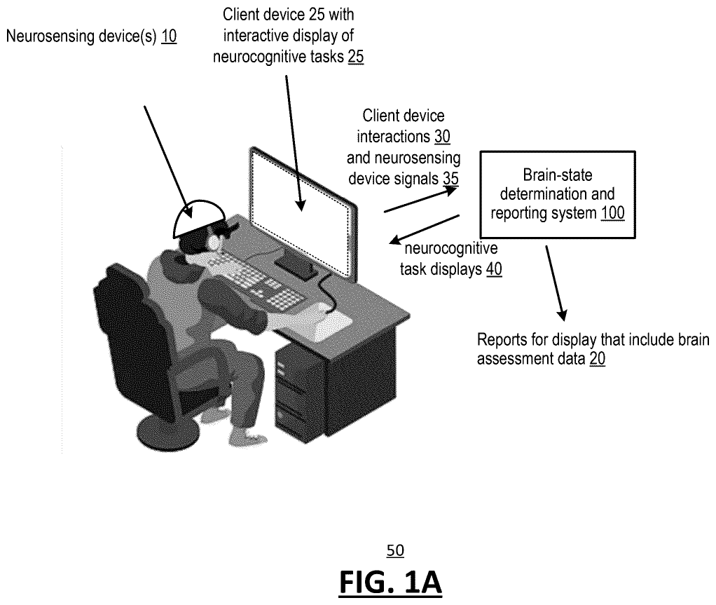

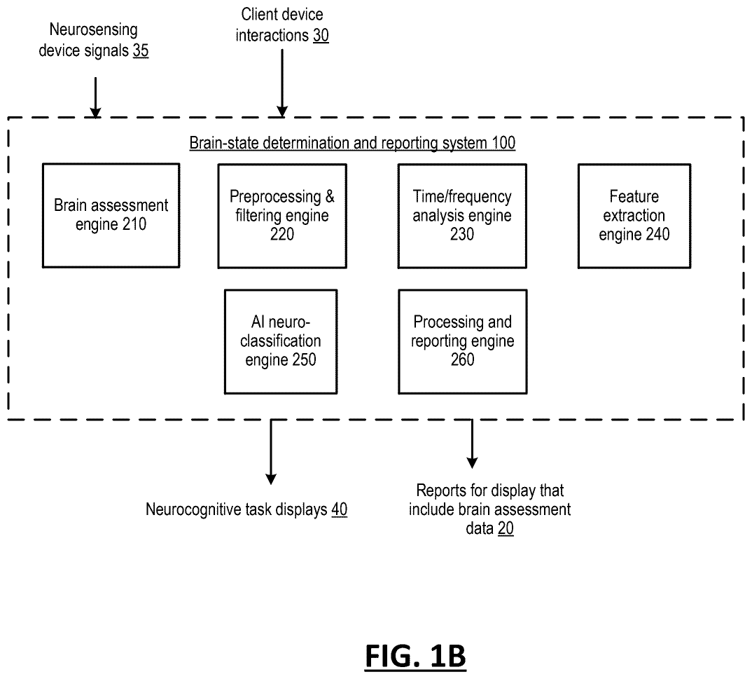

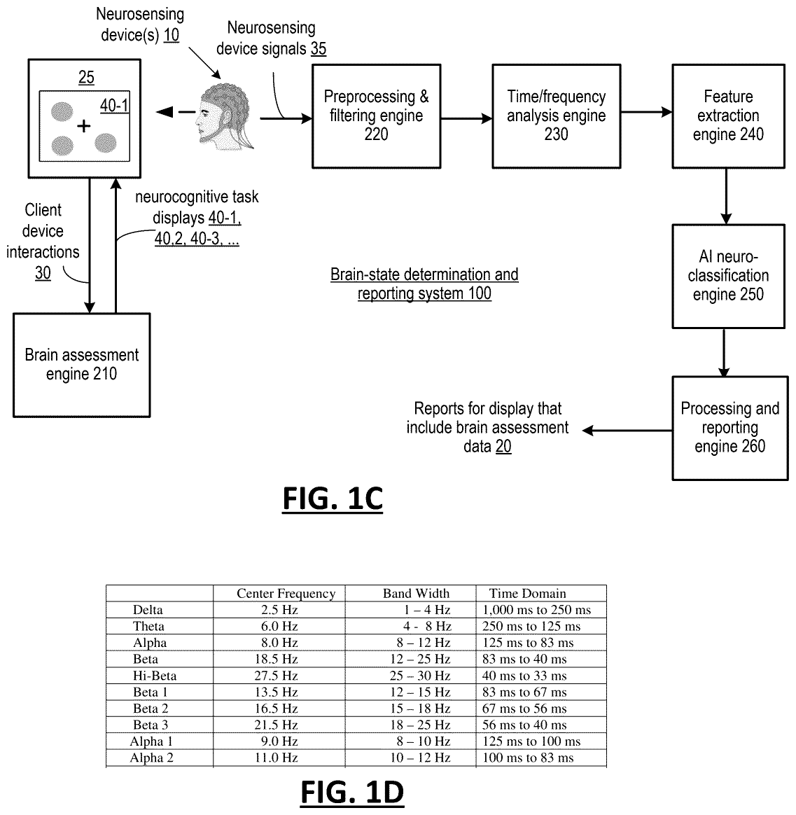

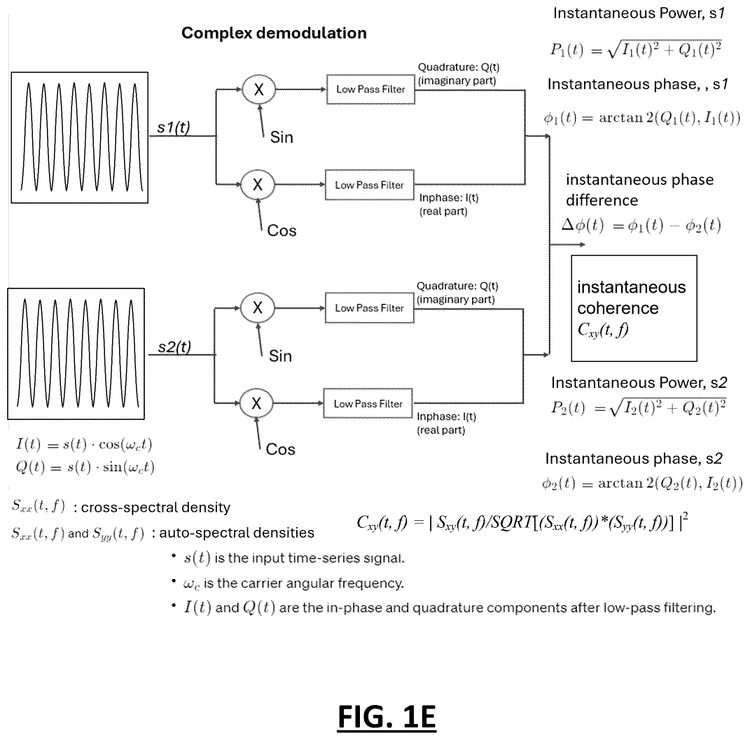

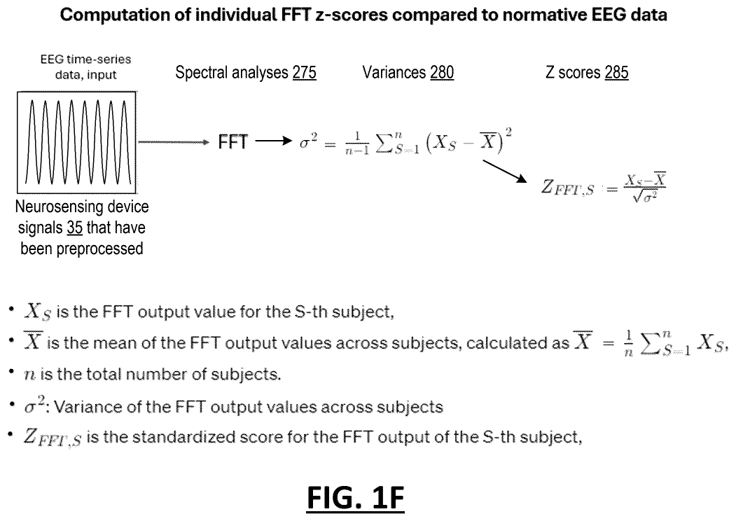

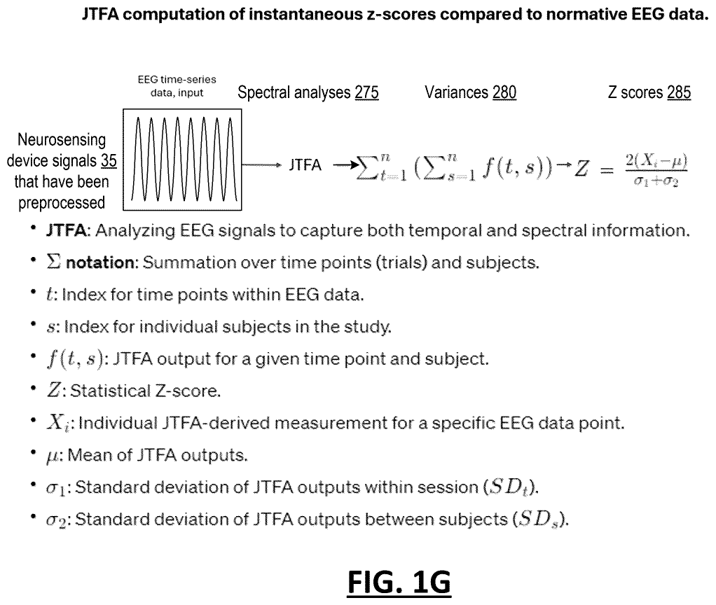

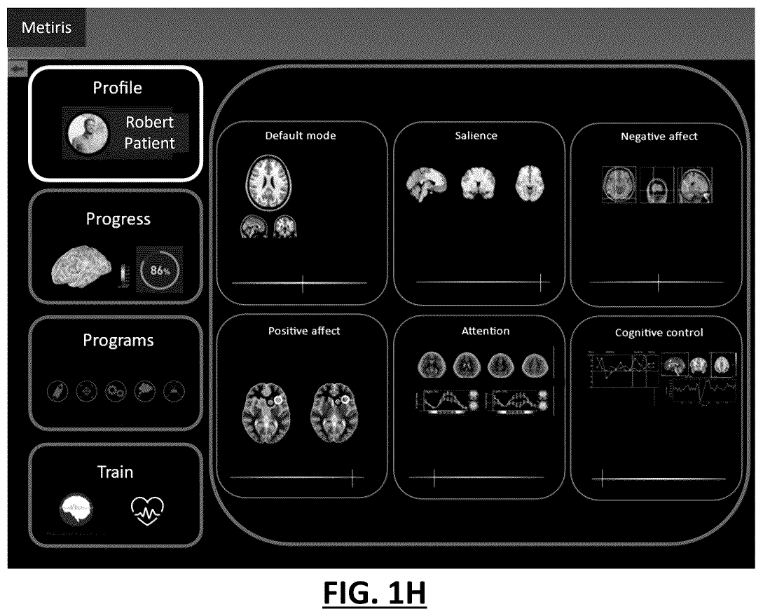

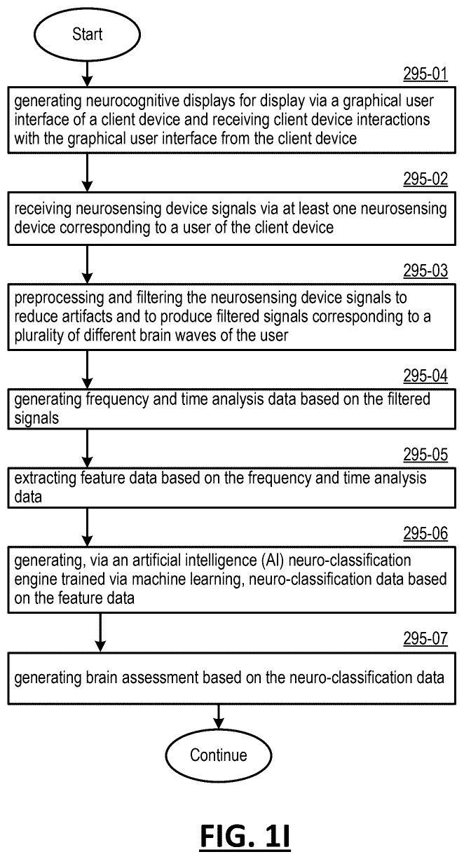

FIG. 1A is a schematic block diagram of an example of a processing system that generates brain assessment data;

FIG. 1B is a schematic block diagram of an example of a brain-state determination and processing system;

FIG. 1C is a schematic pictorial flow diagram of an example of a brain-state determination and processing system;

FIG. 1D is an example of a table of brain wave frequency and time parameters;

FIG. 1E is a schematic pictorial flow diagram of an example of processing by a brain-state determination and processing system;

FIGS. 1F and 1G are schematic pictorial flow diagrams of example processing by a brain-state determination and processing system;

FIG. 1H is a pictorial diagram of an example screen display;

FIG. 1I presents a flowchart representation of an example method;

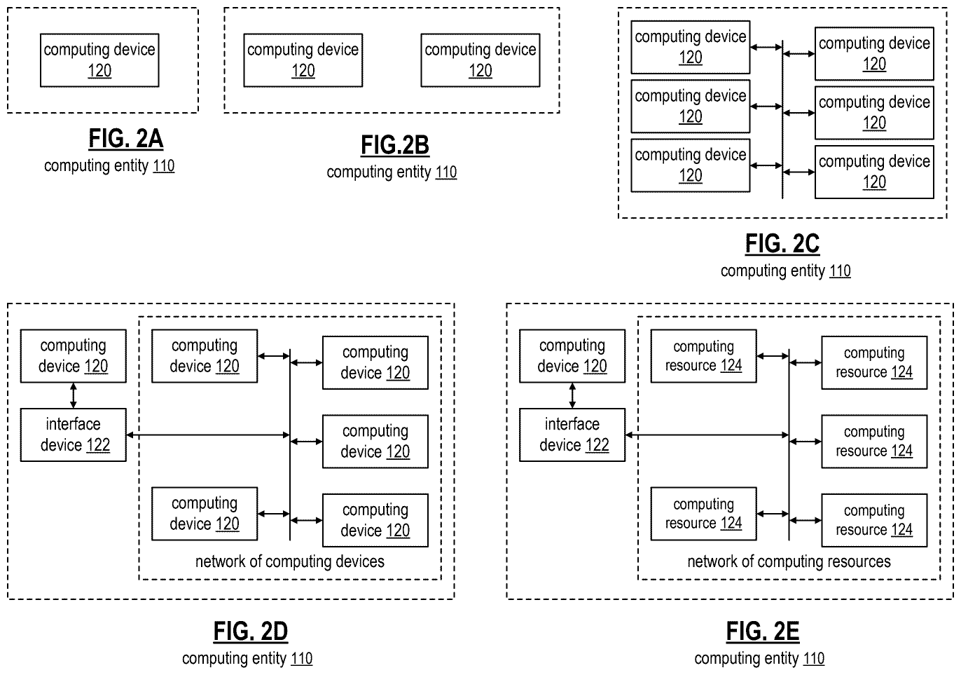

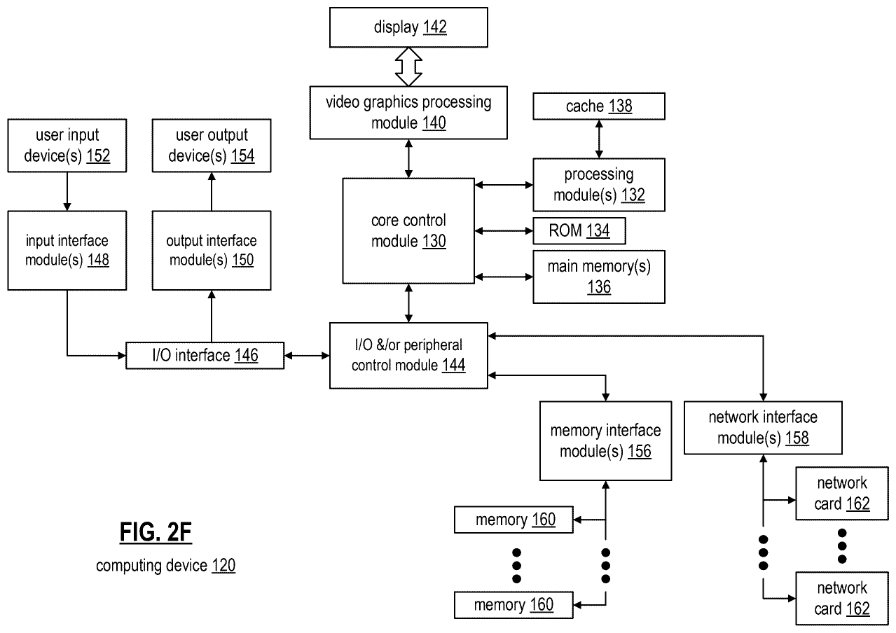

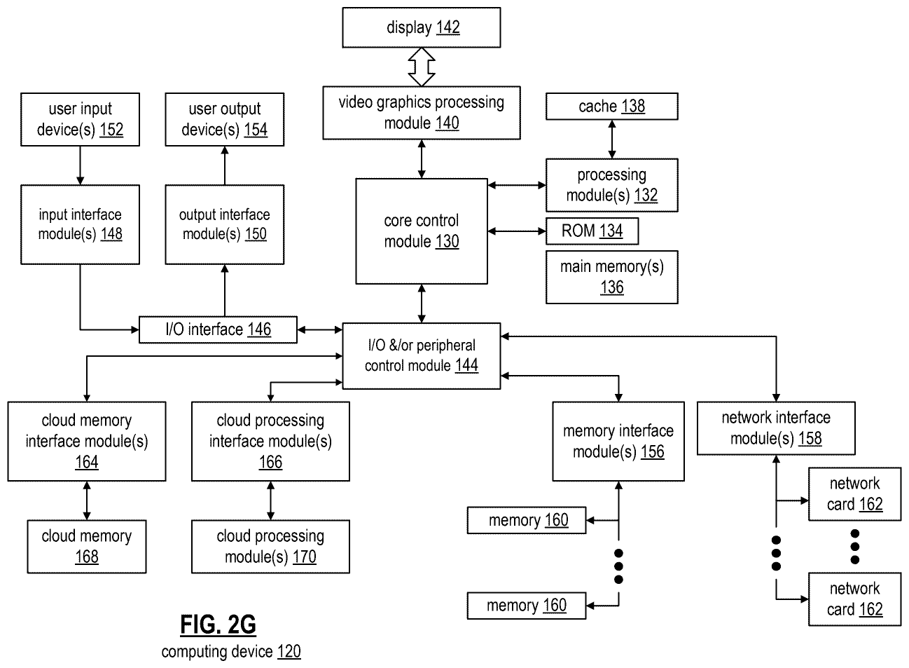

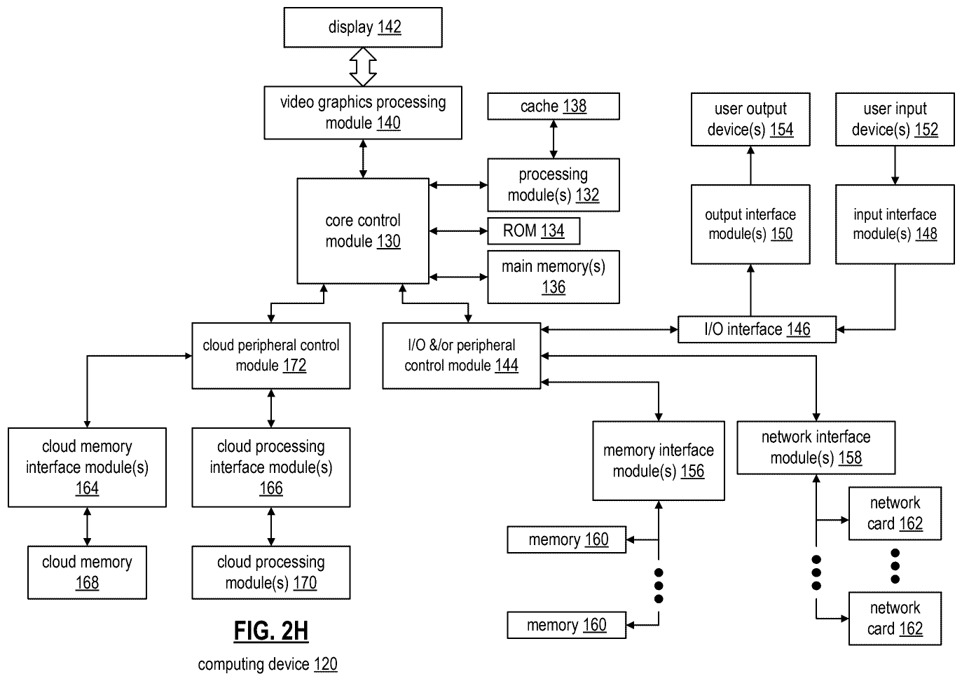

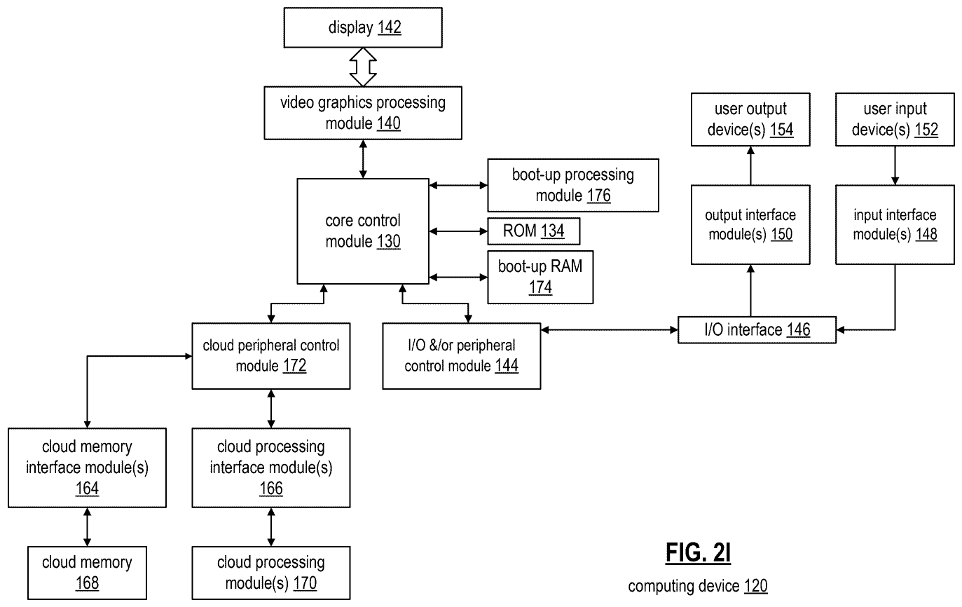

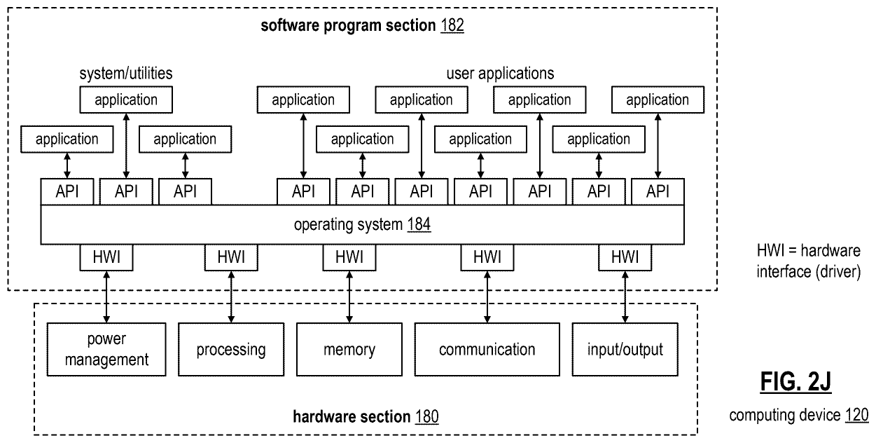

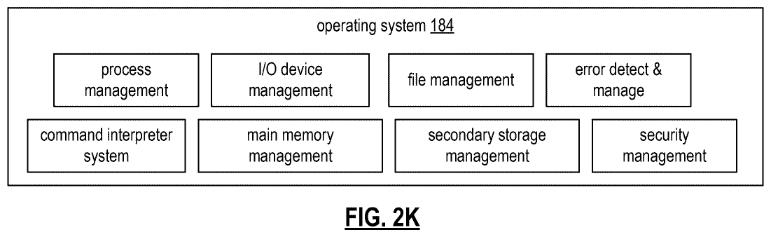

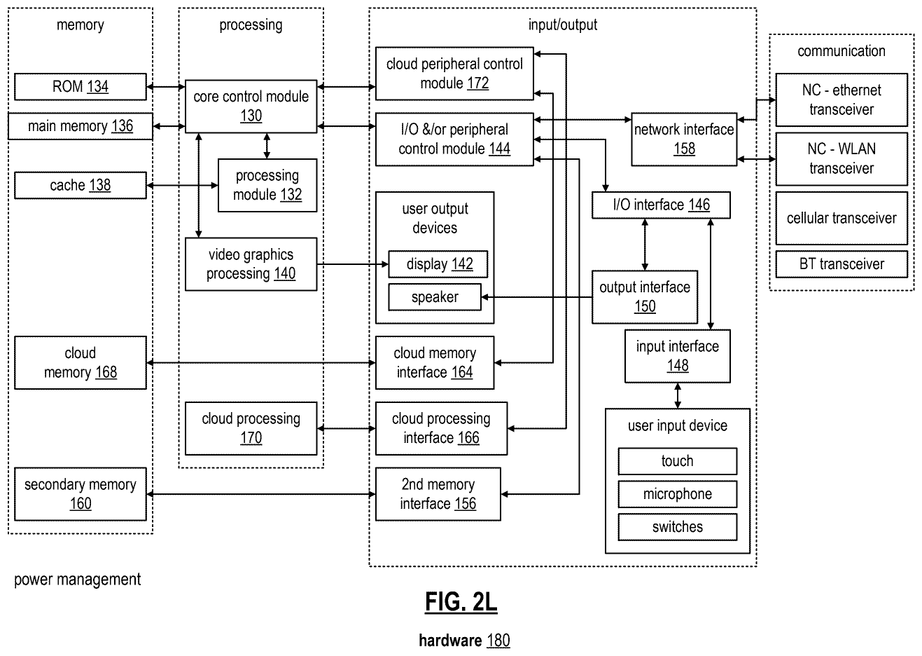

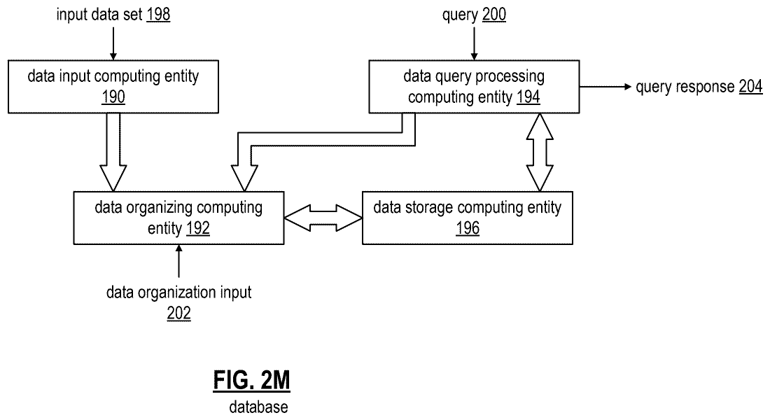

FIGS. 2A through 2E are schematic block diagrams of embodiments of computing entities that are part of an improved computer technology;

FIGS. 2F through 2L are schematic block diagrams of embodiments of computing devices that form at least a portion of a computing entity;

FIG. 2M is a schematic block diagram of an embodiment of a database;

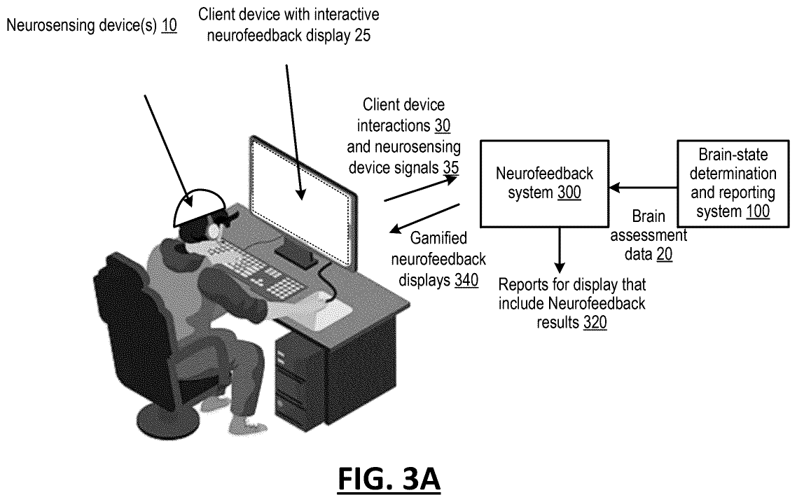

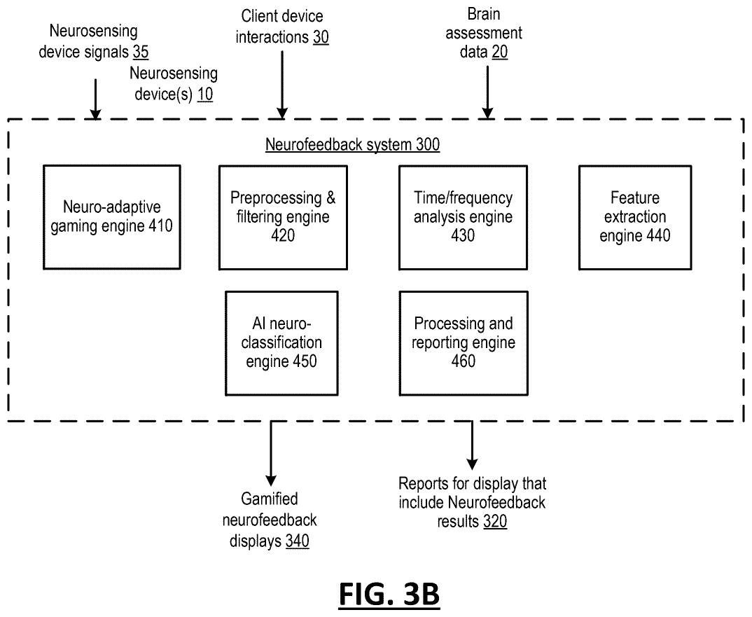

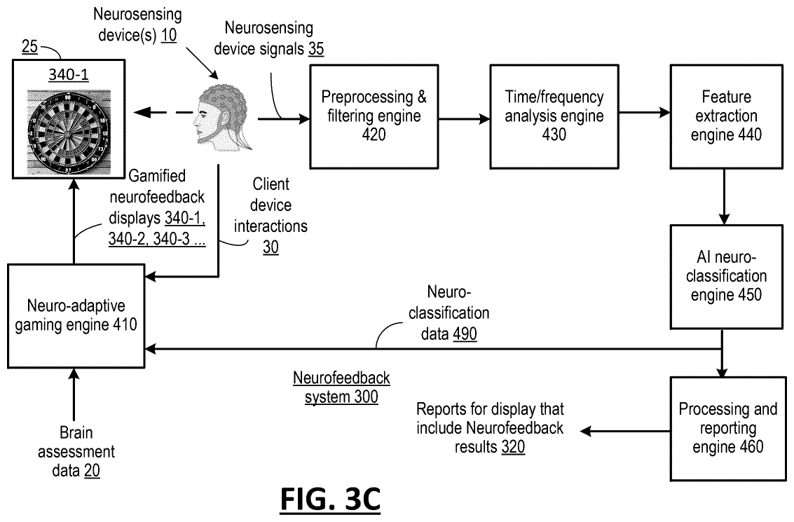

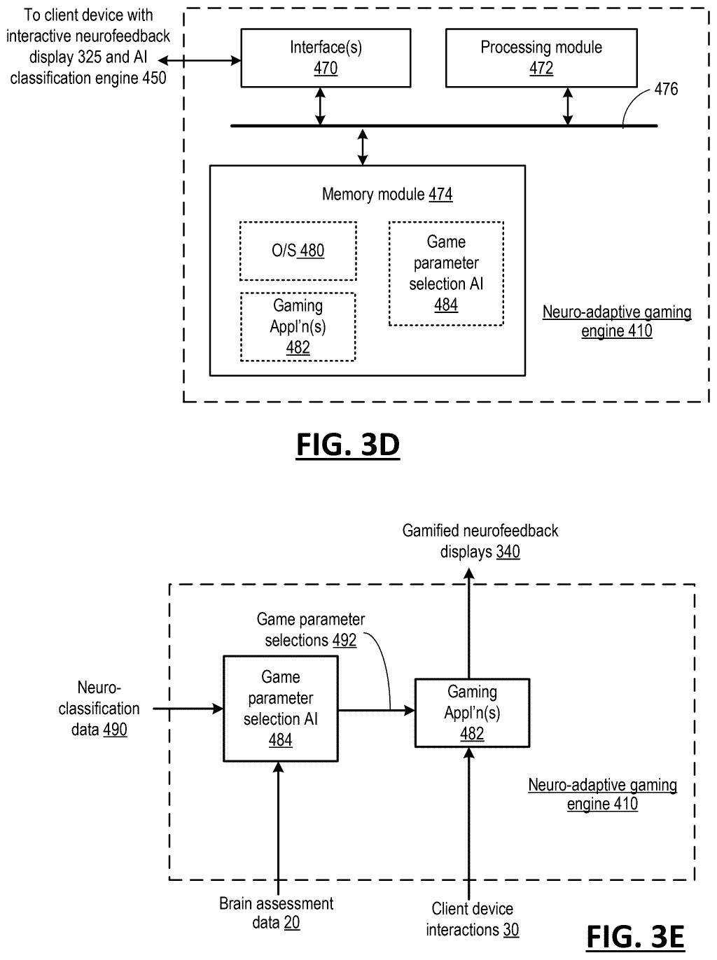

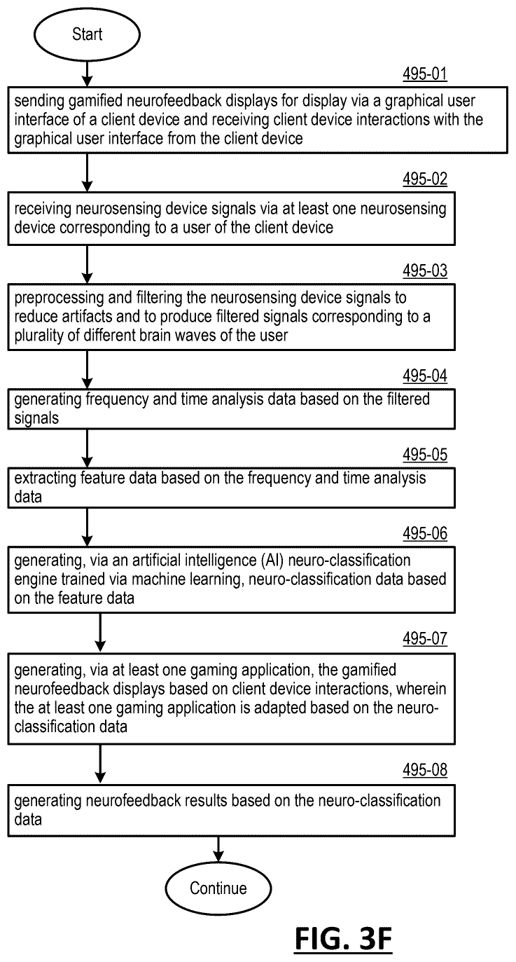

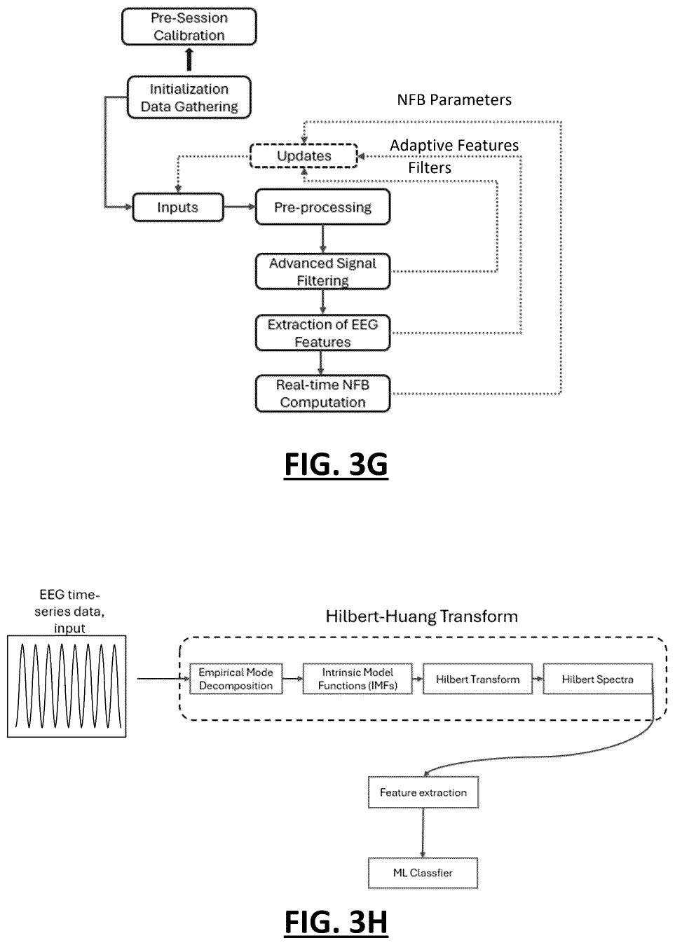

FIG. 3A is a schematic block diagram of an example of a processing system that provides neurofeedback;

FIG. 3B is a schematic block diagram of an example of a neurofeedback system;

FIG. 3C is a schematic pictorial flow diagram of an example of a neurofeedback system;

FIG. 3D is a schematic block diagram of an example of a neuro-adaptive gaming engine;

FIG. 3E is a schematic block diagram of an example of a neuro-adaptive gaming engine; and

FIG. 3F presents a flowchart representation of an example method.

FIG. 3G presents a processing flow representation of a further example of portions of a neurofeedback system.

FIG. 3H presents a processing flow representation of a further example of portions of a neurofeedback system.

### DETAILED DESCRIPTION OF THE INVENTION

Electroencephalography (EEG) is a non-invasive technique used to measure and record the electrical activity of the brain. It provides valuable insights into the brain's functioning and has applications in various fields, including neuroscience, clinical diagnostics, and cognitive research. Traditional EEG techniques operate by measuring the electrical activity of the brain by placing electrodes on the scalp. The electrodes detect and record the electrical signals generated by the synchronized firing of neurons in the brain. These signals, known as brainwaves, are categorized into different frequency bands, including delta, theta, alpha, beta, and gamma waves. Each frequency band is associated with specific mental states and cognitive processes.

EEG recordings can be done in various settings, including hospitals, research laboratories, and even in home-based studies using portable devices. In clinical applications, a technician places electrodes on specific locations on the scalp using a conductive gel or paste. The electrodes are connected to an amplifier, which amplifies and filters the signals. The recorded data is then digitized for further analysis by clinicians who can diagnose and monitor various brain disorders such as epilepsy, sleep disorders, and neurodevelopmental conditions. Abnormal patterns in brainwave activity can indicate the presence of neurological abnormalities and help in designing appropriate treatment plans. EEG is also used in neuroscience research to study brain function and cognitive processes. It can provide valuable insights into attention, perception, memory, and emotional processing. EEG recordings can be combined with other neuroimaging techniques, such as functional magnetic resonance imaging (fMRI), to obtain a further understanding of brain activity. Furthermore, Brain-Computer Interfaces (BCIs) can be implemented which can enable direct communication between the brain and external devices. EEG-based BCIs can decode brainwave patterns associated with specific commands or intentions, allowing individuals to control devices using their thoughts. While traditional EEG is a valuable tool, it has certain limitations.

FIG. 1A is a schematic block diagram of an example of a processing system 50 that generates brain assessment data 20. In operation, the brain-state determination and reporting system 100 generates neurocognitive displays 40 that, for example, include a battery of tasks that are calibrated to elicit a range of neural activities reflective of a subject's psychological and cognitive states, and that provide a comprehensive profiling of brain-state variabilities. These neurocognitive displays 40 are presented via a graphical user interface (GUI) of client device 25 that can also generate client device interactions 30 in response to the subject's interactions with the GUI. The client device 25 can be implemented via a laptop, tablet or other personal computing device, a smartphone, smartwatch, AR/VR googles or glasses or other computing system capable of implementing a GUI. The processing system 50 further includes one or more neurosensing devices 10 having electrodes or other sensors that generate neurosensing signals 35 such as EEG signals or other signaling indicative of the subject's brain activity. The neurosensing device(s) 10 can include an array of scalp sensors, a helmet or cap, a headband or other head-ware. Furthermore, neurosensing device(s) 10 can be incorporated into client device 25 and/or other client devices such as a smartphone, smartwatch, AR/VR googles or glasses, headphones, earbuds, etc.

In various examples, the brain-state determination and reporting system 100 controls the presentation of the neurocognitive displays 40 in response to the client device interactions and analyzes the neurosensing device signals 35 over the battery of tasks in order to automatically generate brain assessment data 20 in the form of textual and/or graphical reports that can present or otherwise indicate neuro-classification data that can include or a subject's initial brain assessment, brain-state dynamics and/or neuroprofiles, which can reveal EEG patterns reminiscent of profiles often observed in conditions such as ADD/ADHD, depression, anxiety, brain injury, etc., as documented in scientific literature. These deviations reflect dysregulated brain activity linked to cognitive, motivational, and other neural mechanisms commonly associated with such conditions. Furthermore, the brain assessment data 20 generated by the brain-state determination and reporting system 100 can indicate specific neurofeedback or other training/treatment protocols that can be used further in neurofeedback procedures and/or other brain function treatment and training.

The processing system 50 improves the technology of neurological analysis by providing advanced signal processing techniques to remove, mitigate or otherwise correct for signal artifacts caused by muscle activity, eye movements, and/or environmental noise. The processing system 50 assesses the functional networks and patterns in the brains of clients (which can also be referred to as subjects or users) with cognitive, behavioral or emotional challenges in technically innovative ways.

The processing system 50 further improves the technology of neurological analysis by employing sophisticated and automated methods of analysis of traditional EEG features relating to brain functions and also new EEG features relating to brain functions not previously measured or analyzed. Brain activity characteristics are measured and tracked that are not currently found in other normative EEG databases and analysis techniques are employed to better separate behavior-related system-level function of the brain from noise.

In various examples, the client device 25 and the brain-state determination and reporting system 100 can each be implemented via a computing entity 110 that will be described in greater detail in conjunction with FIGS. 2A-2M that follow. While shown as separate devices the brain-state determination and reporting system 100 can be implemented via a single such as a smartphone, tablet, laptop or other personal computing system associated with a user either alone or in association with a cloud computing environment.

FIG. 1B is a schematic block diagram of an example of a brain-state determination and processing system 100. In the example shown, the brain-state determination and processing system 100 includes a brain assessment engine 210, a preprocessing and filtering engine 220, a time/frequency analysis engine 230, a feature extraction engine 240, an AI neuro-classification engine 250 and a processing and reporting engine 260. As previously discussed, the brain-state determination and reporting system 100 generates neurocognitive displays 40 and brain assessment data 20 based on client device interactions 30 and neurosensing device signals 35. The brain assessment engine 210, preprocessing and filtering engine 220, time/frequency analysis engine 230, feature extraction engine 240, AI neuro-classification engine 250 and processing and reporting engine 260 can be implemented via one or more computing entity 110 that will be described in greater detail in conjunction with FIGS. 2A-2M that follow. Furthermore, the preprocessing and filtering engine 220, time/frequency analysis engine 230, feature extraction engine 240, and AI neuro-classification engine 250 provide a “signal processing flow” that operates to convert neurosensing device signals 35 into the brain assessment data 20, any or all of which can be used for supporting neurofeedback and further for processing into textual and/or graphical reports via the processing and reporting engine 260. Collectively, the preprocessing and filtering engine 220, time/frequency analysis engine 230, feature extraction engine 240, and AI neuro-classification engine 250 can be referred to as engines of the signal processing flow. Furthermore, while shown as separate components, any of the functionalities of the various engines of the brain-state determination and reporting system 100 and particularly the engines of the signal processing flow can overlap, be combined, be subsumed in other components, performed serially or in parallel as will be apparent to one of ordinary skill in the art.

FIG. 1C is a schematic pictorial flow diagram of an example of a brain-state determination and processing system 100. In an example of operation, brain assessment engine 210 operates by generating neurocognitive displays 40 (e.g., a sequence of displays 40-1, 40-2, 40-3 . . . ) for display via a graphical user interface of a client device 25 and receiving client device interactions 30 with the graphical user interface from the client device (e.g., to control the sequence). The preprocessing & filtering engine 220 operates by receiving neurosensing device signals 35 via at least one neurosensing device 10 corresponding to a user of the client device 25 and further by preprocessing and filtering the neurosensing device signals 35 to reduce artifacts and to produce filtered signals corresponding to a plurality of different brain waves of the user.

The time/frequency analysis engine 230 operates by generating frequency and time analysis data based on the filtered signals. The feature extraction engine 240 operates by extracting feature data based on the frequency and time analysis data. The AI neuro-classification engine 250 is trained via machine learning (ML) such as convolutional neural networks (CNNs), recurrent neural networks (RNNs), Long Short-Term Memory (LSTM), Transformer architectures and their variations and/or other AI and operates by generating neuro-classification data based on the feature data. This AI can be specifically optimized for time-series EEG data, enhancing the identification of individual neural patterns and optionally the identification responses for adaptive neurofeedback (e.g., when used in NFB applications). In various examples, these algorithms can be trained to discern and adapt to the unique signatures of neural network activities and variabilities including observations of user interaction and contextual variables. The processing and reporting engine 260 operates by generating brain assessment data based on the neuro-classification data.

In various examples, the brain assessment engine 210, preprocessing and filtering engine 220, time/frequency analysis engine 230, feature extraction engine 240, AI neuro-classification engine 250 and processing and reporting engine 260 is implemented via at least one processor and at least one memory configured to store operational instructions that, when executed by the at least one processor, cause the at least one processor to perform operations that include:
generating neurocognitive displays display via a graphical user interface of a client device, the graphical user interface further configured receive client device interactions via the client device; and
signal processing flow which includes:
   receiving neurosensing device signals via at least one neurosensing device corresponding to a user of the client device;
   preprocessing and filtering the neurosensing device signals to reduce artifacts and to produce filtered signals corresponding to a plurality of different brain waves of the user;
   generating frequency and time analysis data based on the filtered signals;
   extracting feature data based on the frequency and time analysis data;
   generating, via an artificial intelligence (AI) neuro-classification engine trained via machine learning, neuro-classification data based on the feature data; and
   generating brain assessment data based on the neuro-classification data.

In addition or in the alternative to any of the foregoing, the brain assessment data includes one or more of: neuro-classification data, a neurofeedback target, a neurofeedback protocol, a region of weak neural network activity in the user's brain, a brain state of the user's brain, or a neurological condition of the user of the client device.

In addition or in the alternative to any of the foregoing, the neurocognitive displays are configured to evaluate the user's encoding, maintenance/retention, and recall processes in accordance with a Sternberg spatial working memory paradigm.

In addition or in the alternative to any of the foregoing, the neurocognitive displays correspond to a battery of neurocognitive tasks calibrated to elicit a range of neural activities reflective of a user's psychological and cognitive states.

In addition or in the alternative to any of the foregoing, the feature data is extracted based on one or more of: a phase detection or an envelope detection.

In addition or in the alternative to any of the foregoing, the feature data includes one or more of: a signal amplitude, a band power, or an EEG biomarker.

In addition or in the alternative to any of the foregoing, the feature data includes a plurality of Z scores.

In addition or in the alternative to any of the foregoing, the neurosensing device signals includes electroencephalography signals.

In addition or in the alternative to any of the foregoing, the at least one neurosensing device is incorporated in a gaming device of the user.

In addition or in the alternative to any of the foregoing, the preprocessing includes a standardized weighted Low Resolution Brain Electromagnetic Tomography (swLORETA) coupled with kernel-based temporal enhancement (kTE).

In addition or in the alternative to any of the foregoing, the preprocessing is based on one or more of: Dipole Localization Error (DLE), Euclidean Distance (ED), and Dipole Dispersion (DD).

In addition or in the alternative to any of the foregoing, the time frequency analysis includes a complex demodulation using one or more of: a Hilbert-Huang transform or a joint-time-frequency analysis.

In addition or in the alternative to any of the foregoing, the time frequency analysis generates one or more of: a coherence between channels, a coherence between sources, a phase between channels, a phase between sources, a signal amplitude or a signal density.

In addition or in the alternative to any of the foregoing, the filtering includes spatial filtering and bandpass filtering.

In addition or in the alternative to any of the foregoing, the time frequency analysis includes a wavelet-based independent component analysis.

In addition or in the alternative to any of the foregoing, the frequency and time analysis data is generated based on permutation and randomization tests.

In addition or in the alternative to any of the foregoing, the frequency and time analysis data is authenticated based on surrogate data.

Further examples of the brain-state determination and processing system 100, including the use of this system in generating calibrations, NFB protocols, initial brain states, neuroprofiles and/or other brain assessment data 20 used in neurofeedback, as well as many additional functions and features that can be used in various combinations disclosed herein are discussed in conjunction with FIGS. 1D-1H, 2A-2M and 3A-3H that follow.

FIG. 1D presents an example of a table of brain wave frequency and time parameters used in the signal processing by the brain-state determination and processing system 100. While gamma waves are not expressly shown, distinct center frequency, bandwidth and time domain parameters could also be used for this additional type of brain waves. FIG. 1E illustrates a complex demodulation that generates instantaneous power, coherence and phase difference used in an example of the signal processing by the brain-state determination and processing system. FIGS. 1F and 1G are further examples of the signal processing by the brain-state determination and processing system 100 that employs spectral analysis 275 that includes joint time/frequency analysis JTFA and fast Fourier transforms (FFT), computes variances 280 and Z-scores 285. It should be noted that the Z scores included in brain assessment data 20 can be used in neurofeedback (NFB). FIG. 1H is an example of a pictorial diagram of an example screen display corresponding to brain state assessment data 20 that could further be used in conjunction with a neurofeedback system.

FIG. 1I presents a flowchart representation of an example method. In particular, a method is presented for use with a brain-state determination and processing system, other brain assessment system, and/or that can be implemented in conjunction one or more computing entities 110. Step 295-01 includes generating neurocognitive displays for display via a graphical user interface of a client device and receiving client device interactions with the graphical user interface from the client device. Step 295-02 includes receiving neurosensing device signals via at least one neurosensing device corresponding to a user of the client device. Step 295-03 includes preprocessing and filtering the neurosensing device signals to reduce artifacts and to produce filtered signals corresponding to a plurality of different brain waves of the user. Step 295-04 includes generating frequency and time analysis data based on the filtered signals. Step 295-05 includes extracting feature data based on the frequency and time analysis data. Step 295-06 includes generating, via an artificial intelligence (AI) neuro-classification engine trained via machine learning, neuro-classification data based on the feature data. Step 295-07 includes generating brain assessment data based on the neuro-classification data.

In addition or in the alternative to any of the foregoing, the brain assessment data includes one or more of: neuro-classification data, a neurofeedback target, a neurofeedback protocol, a region of weak neural network activity in the user's brain, a brain state of the user's brain, or a neurological condition of the user of the client device.

In addition or in the alternative to any of the foregoing, the neurocognitive displays are configured to evaluate the user's encoding, maintenance/retention, and recall processes in accordance with a Sternberg spatial working memory paradigm.

In addition or in the alternative to any of the foregoing, the neurocognitive displays correspond to a battery of neurocognitive tasks calibrated to elicit a range of neural activities reflective of a user's psychological and cognitive states.

In addition or in the alternative to any of the foregoing, the feature data is extracted based on one or more of: a phase detection or an envelope detection.

In addition or in the alternative to any of the foregoing, the feature data includes one or more of: a signal amplitude, a band power, or an EEG biomarker.

In addition or in the alternative to any of the foregoing, the feature data includes a plurality of Z scores.

In addition or in the alternative to any of the foregoing, the neurosensing device signals includes electroencephalography signals.

In addition or in the alternative to any of the foregoing, the at least one neurosensing device is incorporated in a gaming device of the user.

In addition or in the alternative to any of the foregoing, the preprocessing includes a standardized weighted Low Resolution Brain Electromagnetic Tomography (swLORETA) coupled with kernel-based temporal enhancement (kTE).

In addition or in the alternative to any of the foregoing, the preprocessing is based on one or more of: Dipole Localization Error (DLE), Euclidean Distance (ED), and Dipole Dispersion (DD).

In addition or in the alternative to any of the foregoing, the time frequency analysis includes a complex demodulation using one or more of: a Hilbert-Huang transform or a joint-time-frequency analysis.

In addition or in the alternative to any of the foregoing, the time frequency analysis generates one or more of: a coherence between channels, a coherence between sources, a phase between channels, a phase between sources, a signal amplitude or a signal density.

In addition or in the alternative to any of the foregoing, the filtering includes spatial filtering and bandpass filtering.

In addition or in the alternative to any of the foregoing, the time frequency analysis includes a wavelet-based independent component analysis.

In addition or in the alternative to any of the foregoing, the frequency and time analysis data is generated based on permutation and randomization tests.

In addition or in the alternative to any of the foregoing, the frequency and time analysis data is authenticated based on surrogate data.

FIGS. 2A through 2E are schematic block diagram of embodiments of computing entities that form at least part of an improved computer technology. In particular, these computing entities can be used to implement the property maintenance platform 30, the client devices 20 and/or 25, and/or the property systems 40.

FIG. 2A is schematic block diagram of an embodiment of a computing entity 110 that includes a computing device 120 (e.g., one or more of the embodiments of FIGS. 2F-2L). A computing device may function as a user computing device, a server, a system computing device, a data storage device, a data security device, a networking device, a user access device, a cell phone, a tablet, a laptop, a printer, a game console, a satellite control box, a cable box, etc.

FIG. 2B is schematic block diagram of an embodiment of a computing entity 110 that includes two or more computing devices 120 (e.g., two or more from any combination of the embodiments of FIGS. 2F-2L). The computing devices 120 perform the functions of a computing entity in a peer processing manner (e.g., coordinate together to perform the functions), in a master-slave manner (e.g., one computing device coordinates and the other supports it), and/or in another manner.

FIG. 2C is schematic block diagram of an embodiment of a computing entity 110 that includes a network of computing devices 120 (e.g., two or more from any combination of the embodiments of FIGS. 2F-2L). The computing devices are coupled together via one or more network connections (e.g., WAN, LAN, cellular data, WLAN, etc.) and perform the functions of the computing entity.

FIG. 2D is schematic block diagram of an embodiment of a computing entity 110 that includes a primary computing device (e.g., any one of the computing devices of FIGS. 2F-2L), an interface device (e.g., a network connection), and a network of computing devices 120 (e.g., one or more from any combination of the embodiments of FIGS. 2F-2L). The primary computing device utilizes the other computing devices as co-processors to execute one or more of the functions of the computing entity, as storage for data, for other data processing functions, and/or storage purposes.

FIG. 2E is schematic block diagram of an embodiment of a computing entity 110 that includes a primary computing device (e.g., any one of the computing devices of FIGS. 2F-2L), an interface device (e.g., a network connection) 122, and a network of computing resources 124 (e.g., two or more resources from any combination of the embodiments of FIGS. 2F-2L). The primary computing device utilizes the computing resources as co-processors to execute one or more of the functions of the computing entity, as storage for data, for other data processing functions, and/or storage purposes.

FIGS. 2F-2L are schematic block diagram of embodiments of computing devices that form at least a portion of a computing entity. FIG. 2F is a schematic block diagram of an embodiment of a computing device 120 that includes a plurality of computing resources. The computing resources, which form a computing core, include one or more core control modules 130, one or more processing modules 132, one or more main memories 136, a read only memory (ROM) 134 for a boot up sequence, cache memory 138, one or more video graphics processing modules 140, one or more displays 142 (optional), an Input-Output (I/O) peripheral control module 144, an I/O interface module 146 (which could be omitted if direct connect IO is implemented), one or more input interface modules 148, one or more output interface modules 150, one or more network interface modules 158, and one or more memory interface modules 156.

A processing module 132 is described in greater detail at the end of the detailed description section and, in an alternative embodiment, has a direction connection to the main memory 136. In an alternate embodiment, the core control module 130 and the I/O and/or peripheral control module 144 are one module, such as a chipset, a quick path interconnect (QPI), and/or an ultra-path interconnect (UPI).

The processing module 132, the core module 130, and/or the video graphics processing module 140 form a processing core for the improved computer. Additional combinations of processing modules 132, core modules 130, and/or video graphics processing modules 140 form co-processors for the improved computer for technology. Computing resources 124 of FIG. 2E include one more of the components shown in this Figure and/or in or more of FIGS. 2G through 2L.

Each of the main memories 136 includes one or more Random Access Memory (RAM) integrated circuits, or chips. In general, the main memory 136 stores data and operational instructions most relevant for the processing module 132. For example, the core control module 130 coordinates the transfer of data and/or operational instructions between the main memory 136 and the secondary memory device(s) 160. The data and/or operational instructions retrieved from secondary memory 160 are the data and/or operational instructions requested by the processing module or will most likely be needed by the processing module. When the processing module is done with the data and/or operational instructions in main memory, the core control module 130 coordinates sending updated data to the secondary memory 160 for storage.

The secondary memory 160 includes one or more hard drives, one or more solid state memory chips, and/or one or more other large capacity storage devices that, in comparison to cache memory and main memory devices, is/are relatively inexpensive with respect to cost per amount of data stored. The secondary memory 160 is coupled to the core control module 130 via the I/O and/or peripheral control module 144 and via one or more memory interface modules 156. In an embodiment, the I/O and/or peripheral control module 144 includes one or more Peripheral Component Interface (PCI) buses to which peripheral components connect to the core control module 130. A memory interface module 156 includes a software driver and a hardware connector for coupling a memory device to the I/O and/or peripheral control module 144. For example, a memory interface 156 is in accordance with a Serial Advanced Technology Attachment (SATA) port.

The core control module 130 coordinates data communications between the processing module(s) 132 and network(s) via the I/O and/or peripheral control module 144, the network interface module(s) 158, and one or more network cards 162. A network card 160 includes a wireless communication unit or a wired communication unit. A wireless communication unit includes a wireless local area network (WLAN) communication device, a cellular communication device, a Bluetooth device, and/or a ZigBee communication device. A wired communication unit includes a Gigabit LAN connection, a Firewire connection, and/or a proprietary computer wired connection. A network interface module 158 includes a software driver and a hardware connector for coupling the network card to the I/O and/or peripheral control module 144. For example, the network interface module 158 is in accordance with one or more versions of IEEE 802.11, cellular telephone protocols, 10/100/1000 Gigabit LAN protocols, etc.

The core control module 130 coordinates data communications between the processing module(s) 132 and input device(s) 152 via the input interface module(s) 148, the I/O interface 146, and the I/O and/or peripheral control module 144. An input device 152 includes a keypad, a keyboard, control switches, a touchpad, a microphone, a camera, etc. An input interface module 148 includes a software driver and a hardware connector for coupling an input device to the I/O and/or peripheral control module 144. In an embodiment, an input interface module 148 is in accordance with one or more Universal Serial Bus (USB) protocols.

The core control module 130 coordinates data communications between the processing module(s) 132 and output device(s) 154 via the output interface module(s) 150 and the I/O and/or peripheral control module 144. An output device 154 includes a speaker, auxiliary memory, headphones, etc. An output interface module 150 includes a software driver and a hardware connector for coupling an output device to the I/O and/or peripheral control module 144. In an embodiment, an output interface module 150 is in accordance with one or more audio codec protocols.

The processing module 132 communicates directly with a video graphics processing module 140 to display data on the display 142. The display 142 includes an LED (light emitting diode) display, an LCD (liquid crystal display), and/or other type of display technology. The display has a resolution, an aspect ratio, and other features that affect the quality of the display. The video graphics processing module 140 receives data from the processing module 132, processes the data to produce rendered data in accordance with the characteristics of the display, and provides the rendered data to the display 142.

FIG. 2G is a schematic block diagram of an embodiment of a computing device 120 that includes a plurality of computing resources similar to the computing resources of FIG. 2F with the addition of one or more cloud memory interface modules 164, one or more cloud processing interface modules 166, cloud memory 168, and one or more cloud processing modules 170. The cloud memory 168 includes one or more tiers of memory (e.g., ROM, volatile (RAM, main, etc.), non-volatile (hard drive, solid-state, etc.) and/or backup (hard drive, tape, etc.)) that is remoted from the core control module and is accessed via a network (WAN and/or LAN). The cloud processing module 170 is similar to processing module 132 but is remote from the core control module and is accessed via a network.

FIG. 2H is a schematic block diagram of an embodiment of a computing device 120 that includes a plurality of computing resources similar to the computing resources of FIG. 2G with a change in how the cloud memory interface module(s) 164 and the cloud processing interface module(s) 166 are coupled to the core control module 130. In this embodiment, the interface modules 164 and 166 are coupled to a cloud peripheral control module 172 that directly couples to the core control module 130.

FIG. 2I is a schematic block diagram of an embodiment of a computing device 120 that includes a plurality of computing resources, which includes include a core control module 130, a boot up processing module 176, boot up RAM 174, a read only memory (ROM) 134, a one or more video graphics processing modules 140, one or more displays 48 (optional), an Input-Output (I/O) peripheral control module 144, one or more input interface modules 148, one or more output interface modules 150, one or more cloud memory interface modules 164, one or more cloud processing interface modules 166, cloud memory 168, and cloud processing module(s) 170.

In this embodiment, the computing device 120 includes enough processing resources (e.g., module 176, ROM 134, and RAM 174) to boot up. Once booted up, the cloud memory 168 and the cloud processing module(s) 170 function as the computing device's memory (e.g., main and hard drive) and processing module.

FIG. 2J is a schematic block diagram of another embodiment of a computing device 120 that includes a hardware section 180 and a software program section 182. The hardware section 180 includes the hardware functions of power management, processing, memory, communications, and input/output. FIG. 2L illustrates the hardware section 180 in greater detail.

The software program section 182 includes an operating system 184, system and/or utilities applications, and user applications. The software program section further includes APIs and HWIs. APIs (application programming interface) are the interfaces between the system and/or utilities applications and the operating system and the interfaces between the user applications and the operating system 184. HWIs (hardware interface) are the interfaces between the hardware components and the operating system. For some hardware components, the HWI is a software driver. The functions of the operating system 184 are discussed in greater detail with reference to FIG. 2K.

FIG. 2K is a diagram of an example of the functions of the operating system of a computing device 120. In general, the operating system function to identify and route input data to the right places within the computer and to identify and route output data to the right places within the computer. Input data is with respect to the processing module and includes data received from the input devices, data retrieved from main memory, data retrieved from secondary memory, and/or data received via a network card. Output data is with respect to the processing module and includes data to be written into main memory, data to be written into secondary memory, data to be displayed via the display and/or an output device, and data to be communicated via a network care.

The operating system 184 includes the OS functions of process management, command interpreter system, I/O device management, main memory management, file management, secondary storage management, error detection & correction management, and security management. The process management OS function manages processes of the software section operating on the hardware section, where a process is a program or portion thereof.

The process management OS function includes a plurality of specific functions to manage the interaction of software and hardware. The specific functions include:
load a process for execution;
enable at least partial execution of a process;
suspend execution of a process;
resume execution of a process;
terminate execution of a process;
load operational instructions and/or data into main memory for a process;
provide communication between two or more active processes;
avoid deadlock of a process and/or interdependent processes; and
control access to shared hardware components.

The I/O Device Management OS function coordinates translation of input data into programming language data and/or into machine language data used by the hardware components and translation of machine language data and/or programming language data into output data. Typically, input devices and/or output devices have an associated driver that provides at least a portion of the data translation. For example, a microphone captures analog audible signals and converts them into digital audio signals per an audio encoding format. An audio input driver converts, if needed, the digital audio signals into a format that is readily usable by a hardware component.

The File Management OS function coordinates the storage and retrieval of data as files in a file directory system, which is stored in memory of the computing device. In general, the file management OS function includes the specific functions of:
File creation, editing, deletion, and/or archiving;
Directory creation, editing, deletion, and/or archiving;
Memory mapping files and/or directors to memory locations of secondary memory; and
Backing up of files and/or directories.

The Network Management OS function manages access to a network by the computing device. Network management includes
Network fault analysis;
Network maintenance for quality of service;
Network access control among multiple clients; and
Network security upkeep.

The Main Memory Management OS function manages access to the main memory of a computing device. This includes keeping track of memory space usage and which processes are using it; allocating available memory space to requesting processes; and deallocating memory space from terminated processes.

The Secondary Storage Management OS function manages access to the secondary memory of a computing device. This includes free memory space management, storage allocation, disk scheduling, and memory defragmentation.

The Security Management OS function protects the computing device from internal and external issues that could adversely affect the operations of the computing device. With respect to internal issues, the OS function ensures that processes negligibly interfere with each other; ensures that processes are accessing the appropriate hardware components, the appropriate files, etc.; and ensures that processes execute within appropriate memory spaces (e.g., user memory space for user applications, system memory space for system applications, etc.).

The security management OS function also protects the computing device from external issues, such as, but not limited to, hack attempts, phishing attacks, denial of service attacks, bait and switch attacks, cookie theft, a virus, a trojan horse, a worm, click jacking attacks, keylogger attacks, eavesdropping, waterhole attacks, SQL injection attacks, and DNS spoofing attacks.

FIG. 2L is a schematic block diagram of the hardware components of the hardware section 180 of a computing device. The memory portion of the hardware section includes the ROM 134, the main memory 136, the cache memory 138, the cloud memory 168, and the secondary memory 160. The processing portion of the hardware section includes the core control module 130, the processing module 132, the video graphics processing module 140, and the cloud processing module 170.

The input/output portion of the hardware section includes the cloud peripheral control module 172, the I/O and/or peripheral control module 144, the network interface module 158, the I/O interface module 146, the output device interface 150, the input device interface 148, the cloud memory interface module 164, the cloud processing interface module 166, and the secondary memory interface module 156. The IO portion further includes input devices such as a touch screen, a microphone, and switches. The IO portion also includes output devices such as speakers and a display.

The communication portion includes an ethernet transceiver network card (NC), a WLAN network card, a cellular transceiver, a Bluetooth transceiver, and/or any other device for wired and/or wireless network communication.

FIG. 2M is a schematic block diagram of an embodiment of a database that includes a data input computing entity 190, a data organizing computing entity 192, a data query processing computing entity 194, and a data storage computing entity 196. Each of the computing entities is an implementation in accordance with one or more of the embodiments of FIGS. 2A through 2E.

The data input computing entity 190 is operable to receive an input data set 198. The input data set 198 is a collection of related data that can be represented in a tabular form of columns and rows, and/or other tabular structure. In an example, the columns represent different data elements of data for a particular source and the rows corresponds to the different sources (e.g., employees, licenses, email communications, etc.).

If the data set 198 is in a desired tabular format, the data input computing entity 190 provides the data set to the data organizing computing entity 192. If not, the data input computing entity 190 reformats the data set to put it into the desired tabular format.

The data organizing computing entity 192 organizes the data set 198 in accordance with a data organizing input 202. In an example, the input 202 is regarding a particular query and requests that the data be organized for efficient analysis of the data for the query. In another example, the input 202 instructions the data organizing computing entity 192 to organize the data in a time-based manner. The organized data is provided to the data storage computing entity for storage.

When the data query processing computing entity 194 receives a query 200, it accesses the data storage computing entity 196 regarding a data set for the query. If the data set is stored in a desired format for the query, the data query processing computing entity 194 retrieves the data set and executes the query to produce a query response 204. If the data set is not stored in the desired format, the data query processing computing entity 194 communicates with the data organizing computing entity 192, which re-organizes the data set into the desired format.

FIG. 3A is a schematic block diagram of an example of a processing system that provides neurofeedback. Neurofeedback is a form of biofeedback that measures and provides feedback on brainwave activity to help individuals learn to self-regulate their brain function. It is based on the principle of operant conditioning, where individuals are rewarded for desired behaviors. In a traditional neurofeedback session, EEG signals are processed and presented to the individual in real-time through visual or auditory feedback, such as changing colors or sounds. The goal of neurofeedback is to train the brain to produce specific brainwave patterns associated with improved cognitive function and symptom reduction. For example, individuals with ADHD may be trained to increase their focus and attention by producing more beta waves, while those with anxiety may be trained to decrease their stress response by producing more alpha waves.

During a neurofeedback procedure, the subject is encouraged to engage in activities that promote the desired brainwave patterns. As the individual's brainwave activity approaches the target range, they receive positive feedback, such as a pleasant sound or a change in visual display. Over time, the brain learns to self-regulate and produce the desired brainwave patterns without external feedback. Neurofeedback is a non-invasive and safe technique that has been used to address various neurological and psychological conditions, including ADHD, anxiety, depression, and sleep disorders. It is typically conducted in a series of sessions, with each session lasting around 30 to 60 minutes. It's important to note that traditional neurofeedback requires input and control by a clinician who have received proper training in the technique, that assesses the individual's specific needs, creates a customized training plan, and monitors progress throughout the sessions.

The neurofeedback system 300 improves upon prior technologies in several ways. neurofeedback system 300 can operate similarly to brain-state determination and reporting system 100 to analyze client interactions 30 and neurosensing device signals 35. Instead of generating neurocognitive brain displays that are used merely for brain assessment and reporting, the client interactions 30 and neurosensing device signals 35 are used in conjunction brain assessment data 20 corresponding to the particular subject to gamify the neurofeedback process based on the subject's personal brain assessment. While processed EEG signals have been used in the past in BCI for gaming, the neurofeedback system 300 introduces gaming as a methodology for generating customized gamified neurofeedback displays 340 that are presented to the subject via a graphic user interface of client device 25. These gamified neurofeedback displays 340 are generated by one or more game applications having parameters that are selected to help induce desired game states through game play—a process that can be more desirable and entertaining to the subject than traditional techniques that rely on merely simple sounds or changes in visual displays. Furthermore, the neurofeedback system 300 can be configured to generate reports for display that include neurofeedback results 320 indicating changes/improvements in brain-state, neuroprofiles or other brain assessments of the subject.

FIG. 3B is a schematic block diagram of an example of a neurofeedback system. In the example shown, the neurofeedback system 300 includes a neuro-adaptive gaming engine 410, a preprocessing and filtering engine 420, a time/frequency analysis engine 430, a feature extraction engine 440, an AI neuro-classification engine 450 and a processing and reporting engine 460. As previously discussed, the neurofeedback system 300 generates gamified neurofeedback displays 340 and neurofeedback results 320 based on client device interactions 30 and neurosensing device signals 35. The neuro-adaptive gaming engine 410, preprocessing and filtering engine 420, time/frequency analysis engine 430, feature extraction engine 440, AI neuro-classification engine 450 and processing and reporting engine 460 can be implemented via one or more computing entities 110. In particular, while shown as separate components, the functionalities of these “engines” can overlap, be combined, be subsumed in other components, performed serially or in parallel as will be apparent to one of ordinary skill in the art. It will be further noted that preprocessing and filtering engine 420, time/frequency analysis engine 430, feature extraction engine 440, AI neuro-classification engine 450 can operate similarly to corresponding elements from FIGS. 1B and 1C.

FIG. 3C is a schematic pictorial flow diagram of an example of a neurofeedback system 300. In an example of operation, neuro-adaptive gaming engine 410 operates by sending gamified neurofeedback displays 340 (e.g., a sequence of displays 340-1, 340-2, 340-3 . . . ) for display via a graphical user interface of a client device 25 and receiving client device interactions 30 with the graphical user interface from the client device (e.g., to control the subject's interaction with the game). The preprocessing & filtering engine 420 operates by receiving neurosensing device signals 35 via at least one neurosensing device 10 corresponding to a user of the client device 25 and further by preprocessing and filtering the neurosensing device signals 35 to reduce artifacts and to produce filtered signals corresponding to a plurality of different brain waves of the user.

The time/frequency analysis engine 430 operates by generating frequency and time analysis data based on the filtered signals. The feature extraction engine 440 operates by extracting feature data based on the frequency and time analysis data. The AI neuro-classification engine 450 is trained via machine learning and operates by generating neuro-classification data 490 based on the feature data. The processing and reporting engine 460 operates by generating neurofeedback results 320 based on the neuro-classification data 490. In this configuration, brain assessment data 20 can be used by the neuro-adaptive gaming engine 410 to select one or more gaming application(s) and/or parameters (e.g., one or more particular games from a stored library of games, opponents and/or capabilities from a stored library of opponents, one or more levels from a stored library of levels, game characters and/or capabilities from a stored library of game characters, a level of difficulty, a color scheme, a graphics layout including a texture complexity for example and/or other parameters of a game of one or more gaming applications to select a gamified neurofeedback protocol for the particular subject, to build game levels driven by individual needs after analyzing some or all of the brain assessment data 20 and/or to address any particular neuroprofile of the subject including one or more deficiencies in the brain-state of the subject. In various examples, the neuro-adaptive gaming engine 410 provides an adaptive gaming interface that is interactive and responsive to the neurofeedback system and that dynamically adjusts to the user's brain activity, thereby gamifying and optimizing the neurofeedback experience. In various examples, the neuro-adaptive gaming engine 410 operates via customizable operant conditioning reinforcement schedules that adapt to individual user responses, employing variable-ratio reinforcement to maximize engagement and efficacy in neurofeedback training. Further the neuro-classification data 490 can be used in real-time to analyze the results of the neurofeedback, improvements/changes in brain-state of the subject and/or to further adapt/select one or more gaming application(s) and/or parameters of one or more gaming applications used to generate the gamified neurofeedback displays 340.

In the example shown, the neuro-adaptive gaming engine 410 employs a dart game in unity that utilizes a plurality of EEG input features from the neuro classification data 490. In other examples, other types of games including sandbox, real-time strategy, shooters, multiplayer online battle arena and other massive online multiplayer games, role-playing, simulation and sports, puzzlers and party games, action-adventure, survival and horror, platformer and other game types. Depending on the subject's neuroprofile, the number of EEG features involved in the training could vary significantly. Some users might engage with around 10 EEG features, aiming to train these features towards a normative range, like a Z-score of zero. The reinforcement in the game can correspond to movements in this desired/targeted training direction. For others, the number of features could be several hundred. In these cases, AI/ML can prioritize extreme outliers first, gradually moving to less extreme variances. This approach ensures that the neurofeedback training is both efficient and effective.

In various examples, the neuro-adaptive gaming engine 410, preprocessing and filtering engine 420, time/frequency analysis engine 430, feature extraction engine 440, AI neuro-classification engine 450 and processing and reporting engine 460 are implemented via at least one processor and at least one memory configured to store operational instructions that, when executed by the at least one processor, cause the at least one processor to perform operations that include:
sending gamified neurofeedback displays for display via a graphical user interface of a client device and receiving client device interactions with the graphical user interface from the client device;
receiving neurosensing device signals via at least one neurosensing device corresponding to a user of the client device;
preprocessing and filtering the neurosensing device signals to reduce artifacts and to produce filtered signals corresponding to a plurality of different brain waves of the user;
generating frequency and time analysis data based on the filtered signals;
extracting feature data based on the frequency and time analysis data;
generating, via an artificial intelligence (AI) neuro-classification engine trained via machine learning, neuro-classification data based on the feature data;
generating, via at least one gaming application, the gamified neurofeedback displays based on client device interactions, wherein the at least one gaming application is adapted based on the neuro-classification data; and
generating neurofeedback results based on the neuro-classification data.

In addition or in the alternative to any of the foregoing, the at least one gaming application is adapted further based on brain assessment data previously generated based on a brain state assessment of the user of the client device.

In addition or in the alternative to any of the foregoing, the brain assessment data includes one or more of: neuro-classification data, a neurofeedback target, a neurofeedback protocol, a region of weak neural network activity in the user's brain, a brain state of the user's brain, or a neurological condition of the user of the client device.

In addition or in the alternative to any of the foregoing, the brain assessment data is generated by evaluating the user's encoding, maintenance/retention, and recall processes in accordance with a Sternberg spatial working memory paradigm.

In addition or in the alternative to any of the foregoing, the brain assessment data is generated based on a battery of neurocognitive tasks calibrated to elicit a range of neural activities reflective of a user's psychological and cognitive states.

In addition or in the alternative to any of the foregoing, the brain assessment data includes functional localizers and wherein the filtering includes spatial filtering that is calibrated based on the functional localizers.

In addition or in the alternative to any of the foregoing, the at least one gaming application is adapted based game parameter selections generated based on game parameter selection AI trained via machine learning.

In addition or in the alternative to any of the foregoing, the neurosensing device signals includes electroencephalography signals.

In addition or in the alternative to any of the foregoing, the at least one neurosensing device is incorporated in a gaming device of the user.

In addition or in the alternative to any of the foregoing, the preprocessing includes a standardized weighted Low Resolution Brain Electromagnetic Tomography (swLORETA) coupled with kernel-based temporal enhancement (kTE).

In addition or in the alternative to any of the foregoing, the preprocessing is based on one or more of: Dipole Localization Error (DLE), Euclidean Distance (ED), and Dipole Dispersion (DD).

In addition or in the alternative to any of the foregoing, the time frequency analysis includes a complex demodulation using one or more of: a Hilbert-Huang transform or a joint-time-frequency analysis.

In addition or in the alternative to any of the foregoing, the time frequency analysis generates one or more of: a coherence between channels, a coherence between sources, a phase between channels, a phase between sources, a signal amplitude or a signal density.

In addition or in the alternative to any of the foregoing, the filtering includes spatial filtering and bandpass filtering.

In addition or in the alternative to any of the foregoing, the time frequency analysis includes a wavelet-based independent component analysis.

In addition or in the alternative to any of the foregoing, the feature data is extracted based on one or more of: a phase detection or an envelope detection.

In addition or in the alternative to any of the foregoing, the feature data includes one or more of: a signal amplitude, a band power, or an EEG biomarker.

In addition or in the alternative to any of the foregoing, the feature data includes a plurality of Z scores.

In addition or in the alternative to any of the foregoing, the frequency and time analysis data is generated based on permutation and randomization tests.

In addition or in the alternative to any of the foregoing, the frequency and time analysis data is authenticated based on surrogate data.

FIG. 3D is a schematic block diagram of an example of a neuro-adaptive gaming engine 410. In this example, the neuro-adaptive gaming engine 410 includes an interface for communicating with an interactive neurofeedback display 325 and AI classification engine 450, a processing module 472 and a memory module 474 that stores an operating system 480, a library of one or more gaming applications 482 having selectable gaming parameters, and instructions corresponding to game parameter selection AI 484. FIG. 3E is a schematic block diagram of an example of a neuro-adaptive gaming engine 410. In an example of operation, a neurofeedback session can be initialized based on brain assessment data 20 corresponding to the subject to allow the game parameter selection AI 484 to select game parameter selections 492 best suited for the subject's particular brain-state targets for improvement. The game parameter selection AI 484 can be trained via machine learning based on a set of training data that indicates how differing games and/or gaming parameters perform in improving past subjects' brain states with differing instances of brain assessment data 20. When neuro-classification data 490 is received during a neurofeedback session, the game parameter selection AI 484 can, in real-time, adapt the game parameter selections 492 based on the actual brain state results. Again, the game parameter selection AI 484 can be trained via machine learning based on a set of training data that indicates how differing games and/or gaming parameters perform in improving past subjects' brain states based on the changes in actual neuro-classifications 490 during a neurofeedback session.

FIG. 3F presents a flowchart representation of an example method. presents a flowchart representation of an example method. In particular, a method is presented for use with a neurofeedback system, and/or that can be implemented in conjunction one or more computing entities 110. Step 495-01 includes sending gamified neurofeedback displays for display via a graphical user interface of a client device and receiving client device interactions with the graphical user interface from the client device. Step 495-02 includes receiving neurosensing device signals via at least one neurosensing device corresponding to a user of the client device. Step 495-03 includes preprocessing and filtering the neurosensing device signals to reduce artifacts and to produce filtered signals corresponding to a plurality of different brain waves of the user. Step 495-04 includes generating frequency and time analysis data based on the filtered signals.

Step 495-05 includes extracting feature data based on the frequency and time analysis data. Step 495-06 includes generating, via an artificial intelligence (AI) neuro-classification engine trained via machine learning, neuro-classification data based on the feature data. Step 495-07 includes generating, via at least one gaming application, the gamified neurofeedback displays based on client device interactions, wherein the at least one gaming application is adapted based on the neuro-classification data. Step 495-08 includes generating neurofeedback results based on the neuro-classification data.

In addition or in the alternative to any of the foregoing, the at least one gaming application is adapted further based on brain assessment data previously generated based on a brain state assessment of the user of the client device.

In addition or in the alternative to any of the foregoing, the brain assessment data includes one or more of: neuro-classification data, a neurofeedback target, a neurofeedback protocol, a region of weak neural network activity in the user's brain, a brain state of the user's brain, or a neurological condition of the user of the client device.

In addition or in the alternative to any of the foregoing, the brain assessment data is generated by evaluating the user's encoding, maintenance/retention, and recall processes in accordance with a Sternberg spatial working memory paradigm.

In addition or in the alternative to any of the foregoing, the brain assessment data is generated based on a battery of neurocognitive tasks calibrated to elicit a range of neural activities reflective of a user's psychological and cognitive states.

In addition or in the alternative to any of the foregoing, the brain assessment data includes functional localizers and wherein the filtering includes spatial filtering that is calibrated based on the functional localizers.

In addition or in the alternative to any of the foregoing, the at least one gaming application is adapted based game parameter selections generated based on game parameter selection AI trained via machine learning.

In addition or in the alternative to any of the foregoing, the neurosensing device signals includes electroencephalography signals.

In addition or in the alternative to any of the foregoing, the at least one neurosensing device is incorporated in a gaming device of the user.

In addition or in the alternative to any of the foregoing, the preprocessing includes a standardized weighted Low Resolution Brain Electromagnetic Tomography (swLORETA) coupled with kernel-based temporal enhancement (kTE).

In addition or in the alternative to any of the foregoing, the preprocessing is based on one or more of: Dipole Localization Error (DLE), Euclidean Distance (ED), and Dipole Dispersion (DD).

In addition or in the alternative to any of the foregoing, the time frequency analysis includes a complex demodulation using one or more of: a Hilbert-Huang transform or a joint-time-frequency analysis.

In addition or in the alternative to any of the foregoing, the time frequency analysis generates one or more of: a coherence between channels, a coherence between sources, a phase between channels, a phase between sources, a signal amplitude or a signal density.

In addition or in the alternative to any of the foregoing, the filtering includes spatial filtering and bandpass filtering.

In addition or in the alternative to any of the foregoing, the time frequency analysis includes a wavelet-based independent component analysis.

In addition or in the alternative to any of the foregoing, the feature data is extracted based on one or more of: a phase detection or an envelope detection.

In addition or in the alternative to any of the foregoing, the feature data includes one or more of: a signal amplitude, a band power, or an EEG biomarker.

In addition or in the alternative to any of the foregoing, the feature data includes a plurality of Z scores.

In addition or in the alternative to any of the foregoing, the frequency and time analysis data is generated based on permutation and randomization tests.

In addition or in the alternative to any of the foregoing, the frequency and time analysis data is authenticated based on surrogate data.

Various additional examples are discussed as follows that include functions and features that can be utilized in addition or in the alternative to any of the foregoing.

As previously discussed, Z scores used to generate and/or be included in brain assessment data 20 and/or otherwise used in neurofeedback (NFB). Consider the following example:
A number X of EEG features identified in a paradigm (rest+task) can be trained (based on AI/ML semisupervised training on large dataset)
Mean and standard deviations are computed for selected variables after resting block (e.g., 10 min) using complex demodulation formulas to derive amplitude/power, phase and coherence.
Reward direction for each NFB EEG feature to be trained is set per variable (up or down regulation toward z=0).
For effective training, there should be between 15-25 discrete rewards per minute-a reward is where the X NFB variables all move in the right direction (and cross their unique threshold) which then activates the generation of NFB display to the client that includes brain state assessment data 20.

In various examples, neurofeedback can be calculated as follows:
The “Z adjustment technique” necessitates that a minimum of 70% of the metrics fulfill the all-or-nothing criteria, followed by the computation of the 10-second historical data pertaining to the 30% failing to meet said criteria. These metrics, identified as outliers or extremes, trigger a feedback signal if their trajectory aligns with Z=0. Evaluate the gradient of a 10-second historical data of Z scores and authorize reinforcement if outliers exhibit movement toward Z=0. In various examples, the brain assessment data 20 and/or neurofeedback results 320 included in the feedback screen can be generated as follows.
Encode both power and connectivity metrics separately in the feedback screen. For instance, if you are using “Balloon” to train, the expansion of its volume corresponds to reduction in z-scored power, the rate at which it achieves coherence, and the luminosity of the entire large balloon to phase. Inflating the balloon to generate a reward requires ensuring normalization across all three metrics, provided they are included in the selected protocol. The results of the adaptive gaming AI technologies including game scores and metrics, as well as neuro-classification data 490 can be also integrated into the feedback design.

FIG. 3G presents a signal processing flow representation of a further example of a neurofeedback system, such as neurofeedback system 300. Prior to neurofeedback the subject has gone through an initial/baseline brain assessment and brain assessment data 20 has been generated that includes a NFB protocol calibrated to best train the weak neural network(s) in the brain of the subject. This can be called “Pre-Session Calibration”. This diagram is not a rigid design architecture but rather a guideline for the real-time EEG processing flow as previously described. Furthermore, its components can be implemented as separate software modules and/or deployed on several processing hardware. The pipeline architecture promotes parallel computing of different signal processing steps, which improves real-time performance.

The (optional) pre-session calibration, usually performed right before the real-time experiments, is used to obtain initialization information for real-time processing during NFB sessions.

The initialization information is usually obtained through a preliminary offline training or calibration session but it can also include a priori information based on empirical knowledge about EEG and/or NFB. Depending on the experimental protocol and the selected signal processing techniques, the initialization might include spatial or temporal filters, band power estimates, signal components (i.e., principal or independent components), thresholds, features or NFB targets.

The filtering step includes more elaborate signal processing operations in the spatial and temporal domain. Commonly used spatial filtering techniques include variants of surface Laplacian, common spatial patterns (CSP), or beamforming. More elaborate techniques that aim at EEG source localization and signal decomposition use various independent component analysis (ICA) methods such as wavelet-based ICA that deconstruct complex neural signals into independent components—thereby refining the accuracy of EEG data analysis, inverse modeling such as standardized weighted LORETA with a temporal kernel. Temporal filtering can be based on the spectral analysis of the EEG signals. The majority of the filtering operations require preliminary training to build subject specific filters and/or mathematical models in order to improve the real-time filtering results.

After filtering, predefined EEG features can be extracted. The choice of features highly depends on the NFB protocol. The most common features are extracted from the signal power analysis in the frequency domain. The features are then used for the NFB calculation. Neurofeedback requires fast computation of signals and relayed back to the client. For this, complex demodulation during filtering, e.g., Hilbert-Huang transforms can be employed as shown in FIG. 3H. These filters can be used to compute instantaneous power, coherence and phase using complex demodulation as previously discussed.

Alternatively, or in addition to the methods mentioned above, the signal processing flow conducted by the NFB system can incorporate an optimized weighted frequency complex-valued finite impulse response (wf-cFIR) filter. This filter is specifically customized for neurofeedback applications, enabling precise temporal filtering and instantaneous extraction of EEG rhythms' power and phase. It is designed to enhance fidelity and temporal specificity in the analysis.

In addition or in the alternative to any of the foregoing, the signal processing flow performed by the NFB system can apply advanced statistical methods, including permutation tests and use of surrogate data, to continuously evaluate and refine neurofeedback protocols, informed by real-time performance metrics and adaptive neuroprofile updates.

In addition or in the alternative to any of the foregoing, a neurofeedback protocol can be constituted of parts, like a building blocks, and AI algorithms employed in the neuro-adaptive gaming engine 410 can evaluate a significance of each of these blocks, effectively putting weight on each component, which can drive the user experience/protocol. This can directly influence stimuli/game to achieve best results.

In addition or in the alternative to any of the foregoing, in-game agent behavior and game content, including levels, audio and text, can be modified and presented via the neuro-adaptive gaming engine 410 as conditioned by predetermined user learning patterns. Observations of the neural network model can fuse context to behavioral and neural manifestations in a spatiotemporal manner. Active learning and knowledge distillation can enable rapid fine tuning of the model. The neuro-adaptive gaming engine 410 can generate a neurofeedback protocol by searching through the shared latent representations and/or a concatenated manifold of neural responses, contextual information and in-game behavioral patterns to find pairs of stimuli and neural responses that are appropriate for a particular context. Global and local optimization methods can be used to identify and propose appropriate patterns of in-game alterations.

In addition or in the alternative to any of the foregoing, a neurofeedback protocol be more than just a list of selected values, like spatial filters, frequencies, etc., but also include a vector of values (responsive from a subjects neural network) that would have encoded all the values and their significance. The AI of the neuro-adaptive gaming engine 410 can, in turn, execute that protocol.

In addition or in the alternative to any of the foregoing, the NFB system integrates a Sternberg spatial working memory paradigm during the initial brain assessment, which evaluates encoding, maintenance/retention, and recall processes, each manifesting distinct EEG features that have been validated in peer-reviewed studies related to ADHD. In various examples, the NFB system is configured to observe the P200 response during the fixation phase as an alerting response and the P300 post-probe presentation for working memory updating. In various examples, the NFB system is configured to compute Cronbach's alpha for each block to assess the reliability of these measures.

In addition or in the alternative to any of the foregoing, the NFB system employs time-frequency analysis of trials, noting alpha desynchronization during encoding and probe presentations (aligned with attentional engagement) and alpha synchronization during maintenance (indicative of internalized attention). Theta power increases, which are expected during maintenance and probing phases, can also be monitored.

In addition or in the alternative to any of the foregoing, the brain assessment data 20 includes functional connectivity measures such as coherence and phase locking values, providing a comprehensive view of the neural networks in operation.

In addition or in the alternative to any of the foregoing, the NFB system generates a resting baseline measure, taken prior to neurofeedback training, that is aligns with traditional protocol development methods and is compared against an age-matched normative database. This comparison can reveal characteristic patterns in ADHD, such as excess delta and theta activity in fronto-central regions during rest, which can be targeted for inhibition or “training down” during neurofeedback.

In addition or in the alternative to any of the foregoing, the NFB system improves on prior technologies by integrating tasks that tap into the neural substrates of attention and working memory, which are often areas of impairment in clients with ADHD. This approach allows for the development of an initial neural profile that includes indices of the central executive, salience, and default mode networks' functionality. This neural profile can then be leveraged using AI/ML algorithms to automatically generate personalized neurofeedback training protocols, bridging the gap between cognitive assessment and targeted neurofeedback intervention. By focusing on “condition” relevant tasks, the system aims to directly address the putative neural weaknesses in subjects, thereby enhancing the specificity and effectiveness of the neurofeedback training.

In addition or in the alternative to any of the foregoing, the signal processing flow performed by the NFB system can apply an EEG an artifact removal/cleaning methodology that incorporates IClabel—an ML method trained on approximately 6,500 annotated EEG datasets—enabling the NFB system to differentiate between clean signals and artifacts.

In addition or in the alternative to any of the foregoing, the signal processing flow performed by the NFB system includes wf-cFIR filters that are used to preprocess EEG data, extracting salient features that capture the dynamics of brain rhythms with high fidelity. These features can include instantaneous power, phase information, and other descriptors of EEG rhythms. The features extracted by cFIR filters serve as a refined input for RNNs, which are adept at learning from time-series data. RNNs can detect patterns over time, learning the sequences of brain-state changes that are indicative of certain cognitive or emotional processes. In various examples, RNNs can predict how certain neural patterns might evolve, which can inform the cFIR filters to adapt their parameters in real time, creating a feedback loop that continuously refines the feature extraction process based on ongoing learning. The combination of cFIR filters and RNNs facilitates the development of personalized neurofeedback protocols. The machine learning model can identify which EEG features and brain rhythms are most responsive to neurofeedback in individual patients, allowing for the customization of the neurofeedback signals. The precise feature extraction capabilities of cFIR filters can be used to modulate gaming environments in real time. For example, in a game designed for neurofeedback, as a player's EEG signals approach the desired brain state, the game could become more visually rewarding or advance in level.

In addition or in the alternative to any of the foregoing, the neuroadaptive game engine responds to instantaneous changes in brain activity, as detected by the cFIR filters. For instance, if a player's focus wanes, the game could introduce new elements to recapture attention, based on the immediate feedback. By tying the gaming feedback to the quality of neurofeedback, as processed by the cFIR filters, the system can implement sophisticated operant conditioning strategies. For example, it could use variable-ratio reinforcement schedules that are known to be highly effective in behavior modification.

In various examples, the system integrates EEG with Generative Pre-trained Transformer (GPT) models, inspired by Natural Language Processing techniques, to decode brain signals and their clinical relevance. By leveraging transformer architectures and attention mechanisms, the system can detect subtle EEG patterns efficiently, enabling few-and zero-shot learning. This adaptability reduces reliance on large datasets, improves diagnostic accuracy, and fosters clinical trust. Designed with clinicians in mind, the software features an intuitive drag-and-drop interface, guided workflows, and GPT-powered natural language querying. The system can automate artifact removal (e.g., eye blinks, muscle noise), data preprocessing, and feature extraction, delivering actionable insights quickly and without advanced technical expertise. AI-driven 3D visualizations, such as heatmaps and symptom-brain graphs, provide spatially explicit interpretations that support earlier interventions and better outcomes.

In various examples, various machine learning models are refined through Machine Learning Operations (MLOps), enabling adaptive, context-aware learning. Unlike traditional models needing manual updates, the system can operate via cumulative, anonymized clinical data streams and automated feedback loops to enhance diagnostic precision, identify new patterns, and personalize treatment recommendations over time.

In various examples, the system utilizes Python for rapid ML and AI prototyping, efficient data handling, and robust visualization. By simplifying complex EEG interpretation and continuously refining its models, the system reduces error rates and provides timely clinical feedback. Non-technical healthcare providers in both high-and low-resource settings can thus offer advanced neurobiological diagnostics, bridging gaps in expertise and infrastructure.

Recognizing the issue of accessibility, especially in underserved communities, the supports offline EEG assessments. In various examples, the system is implemented as a Progressive Web App (PWA), the software allows uninterrupted use with automatic synchronization once connectivity is restored. Web3 technologies further enhance patient data management, enabling decentralized storage and ownership. These features ensure secure, continuous, and patient-empowering care, regardless of constraints.

In addition or in the alternative to any of the foregoing, the system combines blockchain, WebRTC, and edge computing to provide secure and efficient mental health services. In various examples, blockchain is employed to encrypt and decentralize Personal Health Records (PHR), ensuring data integrity and privacy. WebRTC enables low-latency data sharing for real-time neurofeedback and remote consultations. Edge computing processes EEG data locally, maintaining operation in low-bandwidth or offline settings, an issue for underserved areas. Additionally, using Progressive Web Apps (PWAs) and Python-optimized models, the system ensures high accuracy and responsiveness, delivering reliable real-time insights despite connectivity challenges.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provide an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, and/or any device that stores digital information. The memory device may be in a form of a solid-state memory, a hard drive memory, cloud memory, thumb drive, server memory, computing device memory, and/or other physical medium for storing digital information.

As applicable, one or more functions associated with the methods and/or processes described herein can be implemented via a processing module that operates via the non-human “artificial” intelligence (AI) of a machine. Examples of such AI include machines that operate via anomaly detection techniques, decision trees, association rules, expert systems and other knowledge-based systems, computer vision models, artificial neural networks, convolutional neural networks, support vector machines (SVMs), Bayesian networks, genetic algorithms, feature learning, sparse dictionary learning, preference learning, deep learning and other machine learning techniques that are trained using training data via unsupervised, semi-supervised, supervised and/or reinforcement learning, and/or other AI. The human mind is not equipped to perform such AI techniques, not only due to the complexity of these techniques, but also due to the fact that artificial intelligence, by its very definition—requires “artificial” intelligence—i.e., machine/non-human intelligence.

As applicable, one or more functions associated with the methods and/or processes described herein can be implemented as a large-scale system that is operable to receive, transmit and/or process data on a large-scale. As used herein, a large-scale refers to a large number of data, such as one or more kilobytes, megabytes, gigabytes, terabytes or more of data that are received, transmitted and/or processed. Such receiving, transmitting and/or processing of data cannot practically be performed by the human mind on a large-scale within a reasonable period of time, such as within a second, a millisecond, microsecond, a real-time basis or other high speed required by the machines that generate the data, receive the data, convey the data, store the data and/or use the data.

As applicable, one or more functions associated with the methods and/or processes described herein can require data to be manipulated in different ways within overlapping time spans. The human mind is not equipped to perform such different data manipulations independently, contemporaneously, in parallel, and/or on a coordinated basis within a reasonable period of time, such as within a second, a millisecond, microsecond, a real-time basis or other high speed required by the machines that generate the data, receive the data, convey the data, store the data and/or use the data.

As applicable, one or more functions associated with the methods and/or processes described herein can be implemented in a system that is operable to electronically receive digital data via a wired or wireless communication network and/or to electronically transmit digital data via a wired or wireless communication network. Such receiving and transmitting cannot practically be performed by the human mind because the human mind is not equipped to electronically transmit or receive digital data, let alone to transmit and receive digital data via a wired or wireless communication network.

As applicable, one or more functions associated with the methods and/or processes described herein can be implemented in a system that is operable to electronically store digital data in a memory device. Such storage cannot practically be performed by the human mind because the human mind is not equipped to electronically store digital data.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A neurofeedback system comprises:
at least one processor; and
at least one memory configured to store operational instructions that, when executed by the at least one processor, cause the at least one processor to perform operations that include:
sending gamified neurofeedback displays for display via a graphical user interface of a client device and receiving client device interactions with the graphical user interface from the client device;
receiving neurosensing device signals via at least one neurosensing device corresponding to a user of the client device;
preprocessing and filtering the neurosensing device signals to reduce artifacts and to produce filtered signals corresponding to a plurality of different brain waves of the user;
generating frequency and time analysis data based on the filtered signals;
extracting feature data based on the frequency and time analysis data;
generating, via an artificial intelligence (AI) neuro-classification engine trained via machine learning, neuro-classification data based on the feature data;
generating, via at least one gaming application, the gamified neurofeedback displays based on client device interactions, wherein the at least one gaming application is adapted based on the neuro-classification data; and
generating neurofeedback results based on the neuro-classification data.

2. The neurofeedback system of claim 1, wherein the at least one gaming application is adapted further based on brain assessment data previously generated based on a brain state assessment of the user of the client device.

3. The neurofeedback system of claim 2, wherein the brain assessment data includes one or more of: neuro-classification data, a neurofeedback target, a neurofeedback protocol, a region of weak neural network activity in the user's brain, a brain state of the user's brain, or a neurological condition of the user of the client device.

4. The neurofeedback system of claim 2, wherein the brain assessment data is generated by evaluating the user's encoding, maintenance/retention, and recall processes in accordance with a Sternberg spatial working memory paradigm.

5. The neurofeedback system of claim 2, wherein the brain assessment data is generated based on a battery of neurocognitive tasks calibrated to elicit a range of neural activities reflective of a user's psychological and cognitive states.

6. The neurofeedback system of claim 2, wherein the brain assessment data includes functional localizers and wherein the filtering includes spatial filtering that is calibrated based on the functional localizers.

7. The neurofeedback system of claim 1, wherein the at least one gaming application is adapted based game parameter selections generated based on game parameter selection AI trained via machine learning.

8. The neurofeedback system of claim 1, wherein the neurosensing device signals includes electroencephalography signals.

9. The neurofeedback system of claim 8, wherein the at least one neurosensing device is incorporated in a gaming device of the user.

10. The neurofeedback system of claim 1, wherein the preprocessing includes a standardized weighted Low Resolution Brain Electromagnetic Tomography (swLORETA) coupled with kernel-based temporal enhancement (kTE).

11. The neurofeedback system of claim 10, wherein the preprocessing is based on one or more of: Dipole Localization Error (DLE), Euclidean Distance (ED), and Dipole Dispersion (DD).

12. The neurofeedback system of claim 1, wherein the time frequency analysis includes a complex demodulation using one or more of: a Hilbert-Huang transform or a joint-time-frequency analysis.

13. The neurofeedback system of claim 1, wherein the time frequency analysis generates one or more of: a coherence between channels, a coherence between sources, a phase between channels, a phase between sources, a signal amplitude or a signal density.

14. The neurofeedback system of claim 1, wherein the filtering includes spatial filtering and bandpass filtering.

15. The neurofeedback system of claim 1, wherein the time frequency analysis includes a wavelet-based independent component analysis.

16. The neurofeedback system of claim 1, wherein the feature data is extracted based on one or more of: a phase detection or an envelope detection.

17. The neurofeedback system of claim 1, wherein the feature data includes one or more of: a signal amplitude, a band power, or an EEG biomarker.

18. The neurofeedback system of claim 1, wherein the feature data includes a plurality of Z scores.

19. The neurofeedback system of claim 1, wherein the frequency and time analysis data is generated based on permutation and randomization tests or is authenticated based on surrogate data.

20. A brain-state determination and reporting system comprises:
at least one processor; and
at least one memory configured to store operational instructions that, when executed by the at least one processor, cause the at least one processor to perform operations that include:
sending gamified neurofeedback displays for display via a graphical user interface of a client device and receiving client device interactions with the graphical user interface from the client device;
receiving neurosensing device signals via at least one neurosensing device corresponding to a user of the client device;
preprocessing and filtering the neurosensing device signals to reduce artifacts and to produce filtered signals corresponding to a plurality of different brain waves of the user;
generating frequency and time analysis data based on the filtered signals;
extracting feature data based on the frequency and time analysis data;
generating, via an artificial intelligence (AI) neuro-classification engine trained via machine learning, neuro-classification data based on the feature data;
generating brain assessment data based on the neuro-classification data.
